(19) United States
(12) Patent Application Publication
(10) Pub. No.: US 2013/0014769 A1
(43) Pub. Date: Jan. 17, 2013

(54) BACKBOARD COVER AND METHODS OF COVERING A BACKBOARD

(71) Applicant: Gregory CHRISTMAS, Arcadia, CA (US)

(72) Inventor: Gregory CHRISTMAS, Arcadia, CA (US)

(21) Appl. No.: 13/548,495

(22) Filed: Jul. 13, 2012

Related U.S. Application Data
(60) Provisional application No. 61/572,209, filed on Jul. 13, 2011.

(51) Int. Cl. A61B 19/02; B32B 37/14; B32B 38/04; A61F 5/37

(52) U.S. Cl. 128/872; 156/60; 156/252

(57) ABSTRACT

A backboard and backboard cover combination is provided for encapsulating and protecting a backboard from contamination. The backboard is a traditional construction having a top, bottom and rim. Preferably the backboard has one or more holes for ease of carry or for affixing straps. The cover has a length and width sufficient to cover the top and bottom of the backboard. In addition, the cover is made of a flexible, lightweight, chemically inert, mildew, moisture and fungus resistant material. Furthermore, the plastic cover has a cohesive side constructed to adhere to itself, while not adhering to other materials such as the wood or plastic of a backboard. The cover can be applied and encapsulate a backboard by simply crimping the edges of the cover together around the backboard's rim without the requirement for any additional adhesives, staples, velcro, or other fasteners.

### RELATED APPLICATIONS

This application is continuation-in-part of co-pending U.S. Provisional Patent Application Ser. No. 61/572,209 filed Jul. 13, 2011.

### BACKGROUND OF THE INVENTION

The present invention relates to patient handling devices. More particularly, the present invention relates to covers for backboards, also commonly referred to as spinal boards, long spinal boards, longboards and spineboards. A backboard is a planar device utilized for handling patients primarily in pre-hospital trauma care by ambulance services and other emergency personnel. Backboards typically have a top planar surface and a parallel bottom planar surface with a rim forming the periphery of the top and bottom surfaces. In addition, backboards are rigid to provide support during movement of a patient having suspected spinal or limb injuries. Backboards can come in a variety of sizes. However, typical backboards are 6 feet long and 15-18 inches wide. Pediatric backboards are typically 48 inches long and 12 inches wide. These backboards are typically 0.75-2.5 inches thick. Backboards are typically made of wood or plastic. More recently, plastic backboards have become more popular as wood backboards typically require a higher level maintenance to keep them in operable condition and to protect them from cracks and other imperfections that can collect bacteria.

The harboring of bacteria and other contaminants upon a backboard has become a more recognized problem. It has been found that backboards are not cleaned sufficiently after each patient use leaving contaminants such as bodily fluids, microbes, bacteria, and adhesive residue from adhesive tape and cervical immobilization devices. Moreover, backboards are often stacked atop one another such as within an ambulance thereby raising the potential of transferring contaminants between them.

Attempts have been made to provide covers for backboards to protect the backboards from bacterial hazards and the like. For example, U.S. Pat. No. 8,141,555 describes a backboard cover. Unfortunately, the backboard cover described in this patent is complicated to use and expensive to manufacture. Furthermore, the backboard cover suffers the deficiencies that it only partially covers a backboard and loosely secures the cover to the backboard, and potentially allowing bodily fluids, bacteria, and the like to contaminate the backboard through these uncovered areas. Also, this loose covering can be accidentally removed or uncovered in strong winds and poor weather conditions, or when placed in water or submerged in water, or through certain movements when transferring a patient on or off the board.

Accordingly, it would be desirable to provide a backboard cover that was easy and simple to manufacture, and inexpensive to purchase. It would be desirable if the backboard cover provided optimal protection against bacteria and the like contaminating a backboard. Moreover, it would be desirable if the backboard cover was simple to apply to the backboard, simple to remove, and disposable.

Furthermore, it would be desirable to provide a backboard cover that fully encapsulated the backboard to prevent contamination to any area of the board, to stay in place in extreme wind and weather conditions, and when submerged into water, and that is simple to apply, simple to manufacture, and inexpensive to purchase.

### SUMMARY OF THE INVENTION

The present invention is directed to a backboard cover and the method of covering and encapsulating a backboard.

The cover is for covering a traditional backboard made of wood or plastic. The backboard may have any dimensions which are substantially planar for supporting and carrying a patient. However, it is anticipated that the backboard cover of the present invention be utilized primarily for traditional backboards having dimensions 5-7 feet long, 1-2 feet in width, and 0.50 inch-3 inches in thickness. The backboard has a substantially planar top surface for supporting and holding a patient in place. In addition, the backboard has a substantially planar bottom surface so that it can be positioned on the ground or other surface without fear of tipping or rolling. The periphery of the top and bottom backboard surfaces define the backboard's rim. Preferably, the backboard has several holes which are near the backboard's rim which allow emergency rescue personnel to project their hands through the holes to hold and carry the backboard. Alternatively, the holes can be used to affix straps.

The present invention includes a cover for covering and encapsulating a backboard. Preferably, the cover is made of a low density lightweight plastic which is chemically inert so as to not tarnish or react to the wood or plastic of a backboard. Furthermore, preferably the backboard cover is sufficiently flexible, or in other words, malleable, so as to allow one to manually wrap the cover around and thereafter encapsulate a backboard. Moreover, the backboard cover of the present invention includes at least one surface which is treated with a cohesive material so as to stick to itself, but not stick to dissimilar materials such as the wood or plastic of a traditional backboard. Accordingly, the backboard cover of the present invention includes a cohesive surface, but one that does not have a traditional adhesive. A preferred backboard cover material is sold by Pregis® Corporation under the trademark Microfoam Cohesive Laminate®. Preferred backboard covers are between 0.02-0.10 inches. Even more preferably, the preferred backboard cover is made of Microfoam Cohesive Laminate® having a thickness of 0.050 inch.

The backboard cover may comprise one, two or more sheets of material. In a first preferred embodiment, the backboard cover includes two sheets with each sheet sized to extend beyond the rim of the top and bottom surfaces of the backboard. Once each sheet is positioned to cover the top or bottom surface of a backboard, the edges of the respective sheets are squeezed together, or in other words crimped, with the cohesive properties of the sheets causing the sheets to adhere together without any additional adhesives or fasteners to thereby encapsulate the backboard.

In an alternative embodiment, the backboard cover includes only a single sheet of material. For this embodiment, the sheet is sized to cover the top and bottom surfaces of the backboard whereby the sheet is folded to cover the top and bottom surfaces. Thereafter, the sheet's edges are manually crimped together to adhere adjacent edges of the cover to encapsulate the backboard.

In still an additional embodiment of the invention, the backboard cover is constructed in the form of an envelope having a central pouch large enough to accept the backboard. For this embodiment, only two edges of the cover are brought together and adhered to encapsulate the backboard.

Where the backboard has holes near the backboard's periphery for making the backboard easy to carry, it is preferred that the cover be punctured at one or more of these “hand holes” so as to allow a person's hand to project through these hand holes so that the backboard can be held and supported by emergency personnel. The cover may be punctured using one's fingers or a tool. However, it is preferred that the cover be made of a sufficiently low strength material such that a person can puncture the cover by simply projecting their fingers through the cover at the hand holes. Thereafter, the opposed cover edges within the hand holes can be brought together to engage and adhere to cover the hole rims.

It is an object of the invention to provide a cover for backboards which protects a backboard from bodily fluids, bacteria and the like.

It is also an object of the invention to provide a backboard cover that is easy to apply and sufficiently inexpensive that a new backboard cover can be used with each subsequent patient.

It is still an additional object of the invention to provide a backboard cover which does not require complicated straps or the like to maintain the backboard cover in place upon a backboard even within extreme environmental conditions such as extreme wind or submerged in water.

Finally, it is an object of the invention to provide a backboard cover which can be easily installed and can be made to include holes to allow persons to grasp the hand holes of a backboard when carrying a patient.

### BRIEF DESCRIPTION OF THE DRAWINGS

FIG. 1 is an exploded perspective view of a preferred embodiment of the backboard cover and backboard combination of the present invention;

FIG. 2 is a perspective view of the backboard and backboard cover of the present invention;

FIG. 3 is a perspective view of the backboard cover and backboard of the present invention wherein a person has utilized their fingers to pierce holes in the backboard cover at the backboard's hand holes;

FIG. 4 is a perspective view of the backboard cover and backboard of the present invention wherein a person has utilized a tool to pierce holes in the backboard cover at the backboard's hand holes;

FIG. 5 is a perspective view of the backboard cover and backboard of the present invention wherein holes have been formed in the backboard cover at the backboard's hand holes and straps have been installed;

FIG. 6 is a perspective view of the backboard cover and backboard of the present invention wherein a patient has been strapped to the backboard;

FIG. 7 is a perspective view of the backboard cover and backboard of the present invention wherein the cover is being removed so as to prevent blood contamination of the backboard;

FIG. 8 is a perspective view of the backboard cover and backboard of the present invention wherein the backboard cover is being discarded;

FIG. 9 is an exploded perspective view of the backboard cover and backboard of the present invention wherein an additional back support member has been applied to the backboard; and

FIG. 10 is a perspective view of the backboard cover, backboard and support member of FIG. 9.

### DETAILED DESCRIPTION OF THE INVENTION

While the present invention is susceptible of embodiment in various forms, as shown in the drawings, hereinafter will be described the presently preferred embodiment of the invention with the understanding that the present disclosure is to be considered as an exemplification of the invention, and it is not intended to limit the invention to the specific embodiments illustrated.

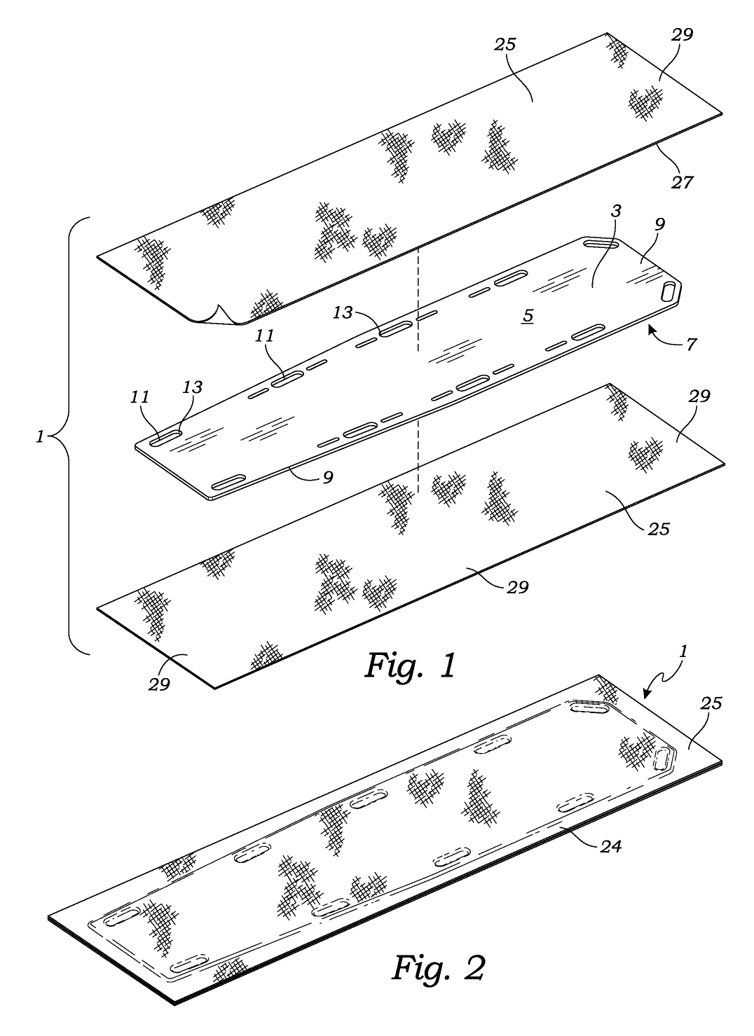

With reference to FIGS. 1-10, the present invention is directed to a backboard and backboard combination 1 wherein the backboard cover 25 can be easily applied to cover the backboard 3, and the backboard cover 25 can be easily removed for disposal. With reference to FIGS. 1, 8 and 9, the cover 25 is intended for use in covering traditional backboards 3 used by emergency medical technicians to transport patients, primarily for use in pre-hospital trauma care. The backboards described herein have a traditional construction, though the present invention is suitable for use with backboards of less traditional construction.

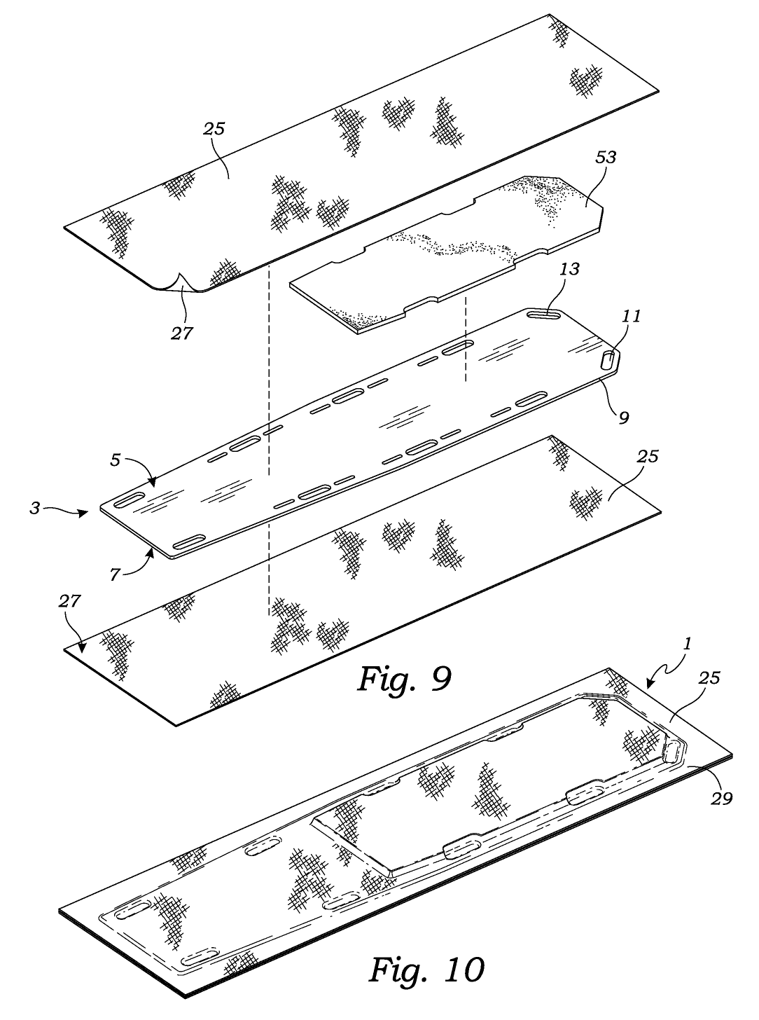

As illustrated in the drawings, a preferred backboard 3 has a top substantially planar surface 5 and a substantially planar bottom surface 7. These surfaces extend horizontally to form the backboards rim 9. The backboard may be made of any material. However, preferred backboard constructions are made of wood or plastic. Preferred backboard dimensions are 6 feet in length, 15-18 inches in width and 0.5-2.5 inches in thickness. As illustrated in FIG. 9, the backboard may include additional back supports 53 to provide additional support for a patient's back and/or neck. The back support member may be made of a firm material for support, or may be made of softer materials, such as rubber or soft foam, to provide comfort to a patient.

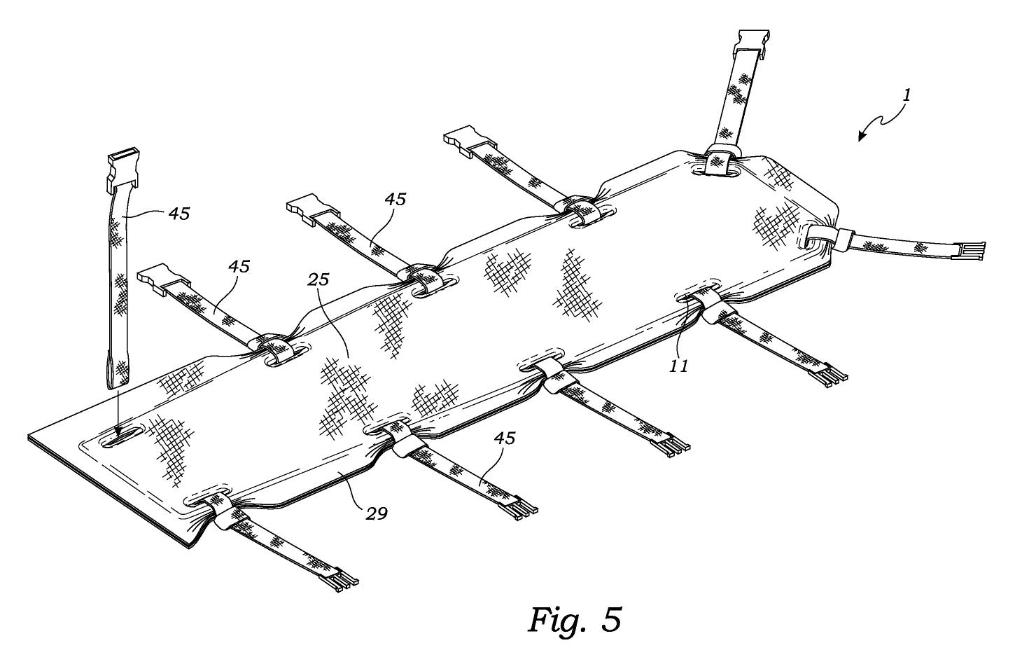

As illustrated in the figures, backboards 3 typically have one or more holes 11 near the backboard's periphery. These holes are typically used for affixing straps 45, as seen in FIG. 5, or for ease in carrying the backboard by emergency medical technicians. For purposes herein, these holes 11 will be referred to herein as “hand” holes wherein the periphery of the holes 11 form the hole rims 13.

The backboard cover 25 of the present invention may be constructed of a single sheet. However, in a preferred embodiment illustrated in FIGS. 1, 7 and 9, the backboard cover is constructed in two sheets. The backboard cover sheets have a length and width greater than the length and width of the backboard 3 so as to be capable of entirely encapsulating the backboard. To provide mildew, moisture and fungus resistance, the backboard cover is preferably made of a plastic, and preferably of a plastic foam such as Microfoam Cohesive Laminate® sold by Pregis® Corporation. The preferred material has a 50 mil. layer of polypropylene foam. On one side of the foam is a laminate of 0.75 polyethylene film. On the opposing side of the polypropylene foam is a cohesive coating capable of sticking to itself, but not sticking to traditional backboard materials such as wood or plastic. Other materials may provide the required flexibility, lightweight and cohesiveness while providing mildew, moisture and fungus resistance. However, Microfoam Cohesive Laminate® is currently considered the preferred cover material.

As illustrated in FIGS. 1 and 2, a top and bottom layer of the cover 25 is positioned to cover the top and bottom sides 5 and 7 of the backboard. As explained above, the cover may be constructed of a single sheet wherein the sheet is folded to provide two portions which cover the top and bottom of the backboard 3. However, two separate sheets are considered the preferred embodiment. As illustrated in FIG. 2, the respective sheets of the cover 25 are brought together so that there cohesive sides engage at the cover's edges 29. Emergency medical technicians or the like then manually squeeze the respective edges together so as to crimp the edges into an adhered state. No additional adhesive or fasteners are necessary to maintain the cover edges 29 together including during normal patient handling. Excess cover material can be removed, such as at the corners, by utilizing scissors or the like. However, it is preferred that the cover be sized so that excess material is minimized and removal of excess material is not necessary for normal handling of the backboard.

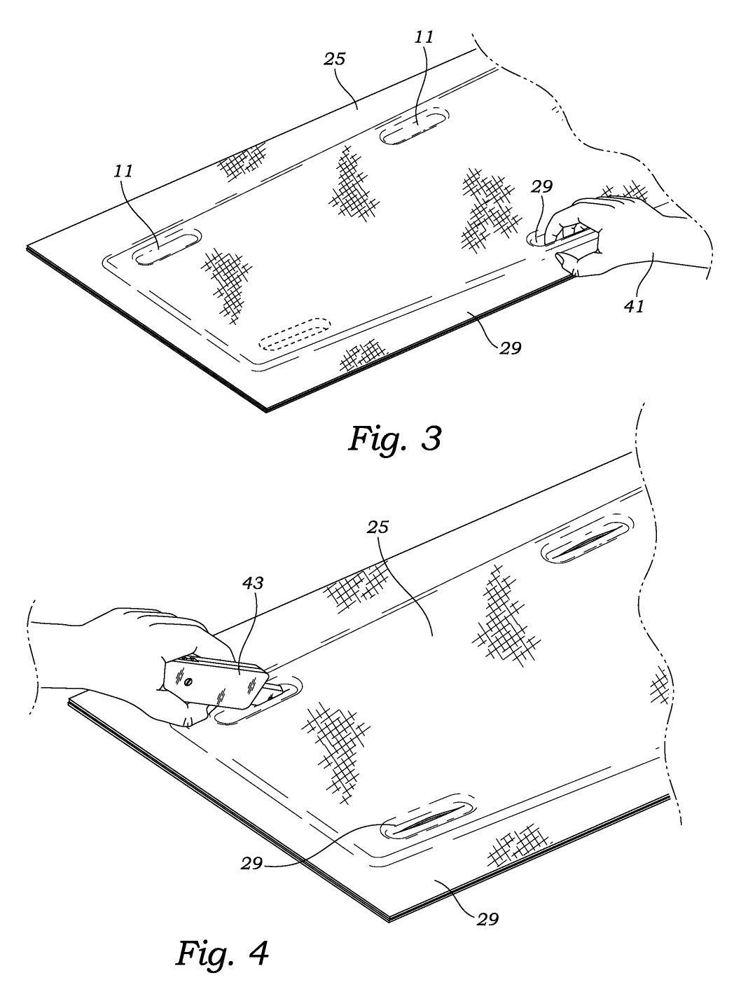

As illustrated in FIGS. 3 and 4, it is preferred that the cover 25 be punctured at one or more hand holes 11. The cover may include perforations in the material made during manufacturing process to assist in emergency medical technicians piercing the cover at the hand holes. However, perforations are not considered necessary for performing the puncturing operation in the field. Moreover, considering the wide variety of backboard constructions, it is not considered ideal to provide perforations in the cover. Instead, the cover material is of sufficiently low strength that a person can puncture the one or more hand holes simply by utilizing their fingers to push through the cover material, such as illustrated in FIG. 3. Alternatively, the person can utilize a tool, such as a knife blade, to puncture the cover at the hand holes more quickly, as illustrated in FIG. 4. Once the cover has been punctured on both sides at the backboard hand holes, one can project the cover into the hand holes at both sides and adhere the cover sheet edges together to encapsulate the hole rims 13 by simply crimping the excess material of the cover so that the cohesive sides engage.

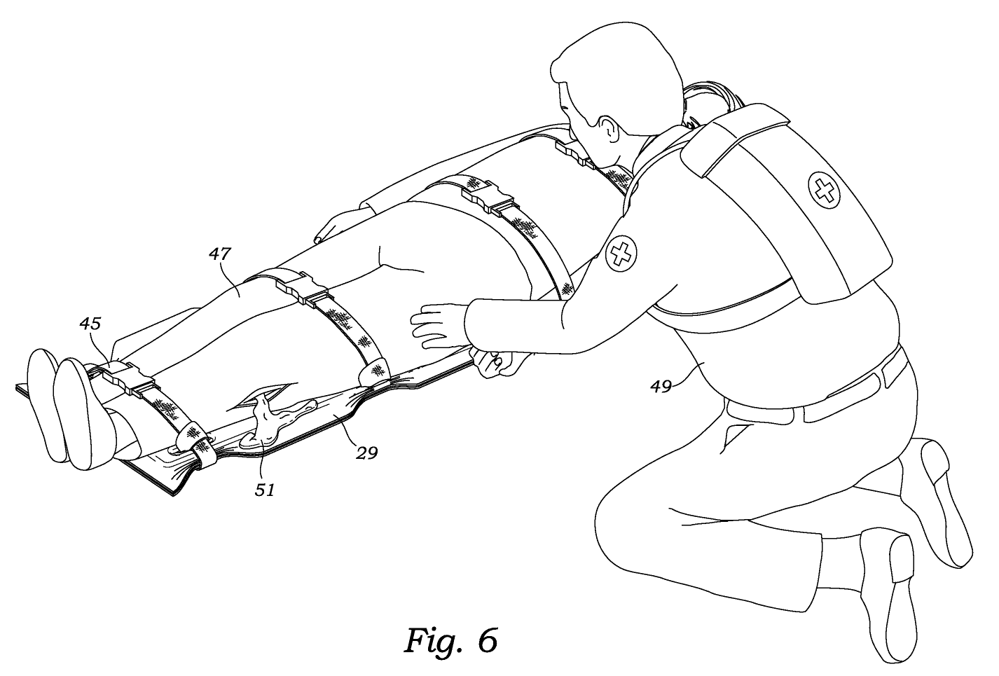

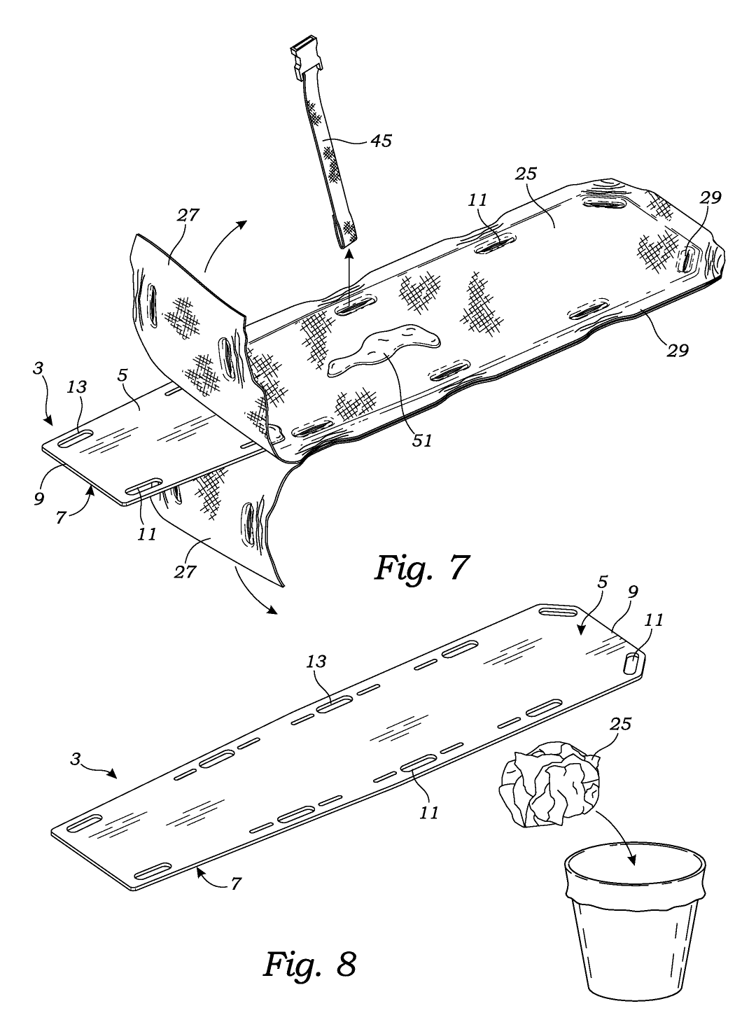

As illustrated in FIGS. 5-10, once the backboard including its hand holes have been entirely encapsulated by the cover, straps 45 can be applied. Emergency medical technicians 49 can support and transport a patient 47 with the backboard and backboard cover combination 1. As illustrated in FIGS. 6 and 7, any bodily fluids such as blood 51, may encounter the exterior of the cover 25. However, the underlying backboard 3 is protected from such bodily fluids. Once a patient has been successfully carried to a desired location, such as a hospital, the straps 45 can be removed, and the blood contaminated cover can be removed from the backboard 3 for disposal.

While several particular forms of the invention have been illustrated and described, it will be apparent that various modifications can be made without departing from the spirit and scope of the invention. Accordingly, it is not intended that the invention be limited except by the following claims. Having described my invention in such terms as to enable a person skilled in the art to understand the invention, recreate the invention and practice it, and having presently identified the presently preferred embodiments thereof,

I claim:

1. A patient handling backboard and backboard cover combination comprising:
a patient handling backboard made of wood or plastic having a top substantially planar surface sized for supporting a patient, a bottom substantially planar surface, a rim forming the periphery of said top and bottom surfaces, and a plurality of hand holes projecting through backboard from said top surface to said bottom surface with said hand holes forming hole rims; and
a cover covering said backboard, said cover made of a plastic having a self adherent surface adhering to itself and have greater adherence to itself than to the wood or plastic of said backboard, said cover having edges and encapsulating said backboard by adhering to itself at said edges.

2. The patient handling backboard and backboard cover combination of claim 1 wherein said self-adherent plastic is a polypropylene sheet coated with self adherent adhesive.

3. The patient handling backboard and backboard cover combination of claim 1 wherein said cover is a low strength material such that a person can puncture said cover with their fingers by projecting their fingers through said cover.

4. The patient handling backboard and backboard cover combination of claim 1 wherein said cover is punctured at one or more of said hand holes and said cover projects into and over said hand holes to cover said hole rims.

5. A method of covering a patient handling backboard comprising the steps of:
providing a patient handling backboard made of wood or plastic having a top substantially planar surface sized for supporting a patient, a bottom substantially planar surface, a rim forming the periphery of the top and bottom surfaces, and a plurality of hand holes projecting through the backboard from the top surface to the bottom surface with the hand holes forming hole rims; and
providing a sheet with edges, the sheet made of plastic having a self adherent surface adhering to itself and have greater adherence to itself than to the wood or plastic of the backboard;
covering the backboard with the sheet; and
encapsulating the backboard with the sheet by adhering sheet edges to the sheet itself.

6. A method of covering a patient handling backboard of claim 5 further comprising the steps of:
manually puncturing the cover at one or more of the hand holes;
projecting the cover into the hand holes to encapsulate said hole rims by adhering the cover sheet edge to the sheet itself.

7. A method of covering a patient handling backboard of claim 5 further comprising the steps of:
manually puncturing the cover at one or more of the hand holes by projecting a person's fingers through the cover at one or more of the backboard's hand holes;
covering the hole rims by adhering a cover sheet edge to an adjacent cover sheet edge.

8. A method of covering a patient handling backboard of claim 5 further comprising the steps of:
manually puncturing the cover at one or more of the hand holes by projecting a tool through the cover at one or more of the backboard's hand holes;
covering the hole rims by adhering a cover sheet edge to an adjacent cover sheet edge.
